(19) United States
(12) Patent Application Publication
(10) Pub. No.: US 2006/0025419 A1
(43) Pub. Date: Feb. 2, 2006

(54) Imidazoquinoxaline compound for the treatment of melanoma

(76) Inventors: Ann Richmond, Brentwood, TN (US); Jinming Yang, Nashville, TN (US); Katayoun Amiri, Nashville, TN (US); Punita Dhawan, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 11/166,791

(22) Filed: Jun. 24, 2005

Related U.S. Application Data
(60) Provisional application No. 60/582,851, filed on Jun. 25, 2004.

(51) Int. Cl. A61K 31/498; G01N 33/574

(52) U.S. Cl. 514/250; 435/7.23

(57) ABSTRACT

The present invention concerns a (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid (BMS-345541) or an analog thereof for the treatment of melanoma cancer. This compound inhibits NFκB activity and expression, and induces apoptosis in melanoma cancer cells. The present invention also provides a method for assaying for the inhibition of melanoma cancer cell growth.

The present invention claims benefit of priority to U.S. Provisional Application Ser. No. 60/582,851, filed Jun. 25, 2004, the entire contents of which are hereby incorporated by reference.

The government owns rights in the present invention pursuant to grant numbers CA56704, CA68485 and 5P30AR41943 each from the National Institutes of Health.

### BACKGROUND OF THE INVENTION

1. Field of the Invention

The present invention relates generally to the fields of cancer biology and cancer therapeutics. More particularly, it concerns the use of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, also referred to herein as BMS-345541 or salts or analogs thereof, in the treatment of melanoma.

2. Description of Related Art

Since the 1940's the incidence of melanoma has doubled every year. Since 1981 the incidence of melanoma has increased 7 percent per year to a rate of 14.3 per 100,000 in 1997. In 2003 there were about 53,000 new diagnosed cases. Melanoma is the most common cancer among people 25 to 29 years of age. More than 80% of skin cancer deaths are due melanoma. Melanoma is the sixth most common cancer in men and the seventh most common in women. Melanoma has a 5 year survival rate of 30 to 40% and often spreads to distant organs such as, liver, bones, brain, lung, etc. This decreases the 5 year survival rate to less than 12%.

Treatment of melanoma poses a great challenge for researchers today due to its resistance to conventional chemotherapeutics and radiation (Smalley and Eisen, 2003; Strauss et al., 2003; Margolin et al., 2002; Chawla-Sarkar et al., 2003). With the increasing number of cases each year, it is imperative to find new targets for the treatment of this cancer. With the realization that different cancers exhibit perturbation of varying biological biochemical pathways, the focus of cancer treatment has turned to more specific, biologically based targets.

In melanoma, aberrant activation of NFκB has been associated with its growth, metastasis, and escape from apoptosis, thus rendering the NFκB pathway as a plausible target for the treatment of melanoma. Previous studies have shown that the persistent activation of NFκB results in production of chemokines such as CXCL8 and CXCL1 that are angiogenic and promote tumorigenesis in melanomas (Richmond, 2002; Richmond et al., 1985; Streit and Detmar, 2003). Furthermore, it was previously shown that the persistent activation of NFκB is mainly due to the constitutive activation of IKK, a major regulator of the NFκB pathway (Yang and Richmond, 2001). Thus, the NFκB pathway, and specifically IKK, are suggested as therapeutic targets in the treatment of melanoma.

### SUMMARY OF THE INVENTION

There remains a need in the art for effective therapeutic agents for treating and/or prevention of cancers such as melanoma. Such therapeutic agents may include the use of synthetic small molecules that target molecules involve in the initiation and/or progression of a melanoma and exhibit minimal toxicity to normal human cells. The present invention is therefore directed to a cancer therapeutic for the treatment and/or prevention of melanoma that overcomes the toxicity, side effects or resistance offered by current chemotherapeutic agents.

Thus, the present invention provides a synthetic compound, (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or salt, or an analog thereof, with minimal toxicity, side effects or resistance, for the treatment of melanoma.

In a particular embodiment, the present invention provides a method of inhibiting growth of a melanoma cancer cell comprising contacting the cell with (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or salt, or an analog thereof. Such a method of inhibiting growth of the melanoma cancer cell further comprises inducing apoptosis, inducing cell cycle arrest, or inducing cell stasis in the melanoma cancer cell.

The melanoma cancer cell as contemplated in the present invention may be located in a cell culture, or tissue culture, or in a mammal such as a human or mouse. The present invention contemplates melanoma cancer cells that are premalignant, malignant, metastatic or multidrug-resistant.

In another particular embodiment of the invention, there is provided a method of inhibiting NFκB activity in a melanoma cancer cell comprising providing to the cell (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or salt, or an analog thereof. Inhibiting NFκB activity in a melanoma cancer cell may further comprise inhibiting expression of NFκB and inducing apoptosis in the cell.

In still another particular embodiment of the invention, there is provided a method of inhibiting tumorigenesis of a melanoma cancer cell comprising contacting the cell with an effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or salt, or an analog thereof. Inhibiting tumorigenesis of a melanoma cancer cell may comprise inducing cytotoxicity in the cell. Inhibiting tumorigenesis of a melanoma cancer cell may also comprise inducing apoptosis in the cell.

It is further contemplated that a second therapeutic agent may be provided in combination with the (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or an analog thereof to a melanoma cancer cell for treatment and/or prevention melanoma. The second agent may be a chemotherapeutic agent, or a radiotherapeutic agent, or an immunotherapeutic agent, or a gene therapy

It is contemplated in the present invention that the (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or pharmaceutically acceptable salt, or an analog thereof and/or the second may be administered to a subject orally, intravenously, intratumorally, intradermally, intramuscularly, intralesionally, percutaneously, subcutaneously, or by inhalation. However, any method of delivering a compound or composition to a subject, as is known to one of ordinary skill in the art, may be employed.

The (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or pharmaceutically acceptable salt, or an analog thereof, of the present invention, may be administered more than once, or before the second therapeutic agent, or after the second therapeutic agent, or at the same time as the second therapeutic agent.

In yet another embodiment of present invention, the second therapeutic agent may be administered intravenously, intradermally, intramuscularly, intraarterially, intralesionally, percutaneously, subcutaneously, or by an aerosol. In a further embodiment, the second therapeutic agent may be administered more than once.

In yet another particular embodiment, the present invention provides a method of assaying for inhibition of melanoma cancer cell growth comprising (a) providing a melanoma cancer cell sample; (b) contacting the melanoma cancer cell sample with an effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or pharmaceutically acceptable salt or an analog thereof; (c) analyzing the cancer cell sample for inhibition of growth; and (d) comparing the inhibition of the cell growth in the cancer cell from step (c) with the inhibition of growth in the melanoma cancer cell in the absence of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or pharmaceutically acceptable salt, or an analog thereof, wherein the difference in growth inhibition represents the growth inhibitory effect of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or pharmaceutically acceptable salt, or an analog thereof or an analog thereof.

The present invention also provides a method of analyzing for growth inhibition is by MTT assay. The assaying methods of the present invention further comprise analyzing for induction of apoptosis by cell count, FACS or TUNEL assay. In yet another embodiment, assaying further comprises analyzing the sample for inhibition or reduction of IKK activity. In still yet another embodiment, assaying further comprises analyzing the sample for inhibition of phosphorylation of IKBα. In still yet a further embodiment, assaying further comprises analyzing a melanoma cancer cell for reduction of expression of a chemokine such as CXCL1 or CXCL8.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E. BMS-345541 inhibits growth of melanoma cells in vitro and in vivo. (FIG. 1A) BMS-345541 inhibition of growth of the cultured SK-Mel-5 cells. 1×10⁵ cells were plated in 6-well plates and cultured in medium containing BMS-345541 at final concentrations of 0, 0.1, 1.0, 10 and 100 μM. After 72 h treatment, cells were photographed. (FIG. 1B) BMS-345541 inhibition of growth of SK-Mel-5 tumors. 2×10⁶ SK-Mel-5 melanoma cells were subcutaneously inoculated into nude mice (n=5 mice per group). When tumors reached an approximate size of 20 mm³, vehicle alone, or BMS-345541 were orally administrated daily at the indicated doses for total 21 days. Tumor size was measured using an electronic digital caliper at intervals of 4 days. Tumor volume was calculated and statistically analyzed. Data shown are representative of two independent experiments. (FIG. 1C) BMS-345541 inhibition of the growth of SK-Mel-5, A375 and Hs294T tumors. Percentage inhibition of tumor growth was calculated from the average tumor size in nude mice (n=10) treated with 75 mg/kg of BMS-345541 for 21 days, versus control tumor size in mice (n=10) treated with vehicle for the indicated type of human melanoma. (FIG. 1D) BMS-345541 distribution in tissues. Mice (n=5) were given a different oral dose of BMS-345541 as indicated. Eight hours after drug administration mouse blood and tumor tissues were collected and BMS-345541 levels were analyzed. (FIG. 1E) BMS-345541 distribution in tissues. Mice (n=3) received an oral dose of 75 mg/kg BMS-345541. BMS-345541 levels in serum (μM, left axis) and tumor tissues (nmoles per gram tumor protein, right axes) were determined at the indicated time points.

FIGS. 2A-2D. BMS-345541 reduces IKK/NF-κB signaling and CXCL1 secretion. (FIG. 2A) Suppression of NF-κB activity in SK-Mel-5 cells. SK-Mel-5 cells were plated in 6-well plates (1.5×10⁵ cells per well) and then cotransfected with 1 μg of HIV long terminal repeat NF-κB-luciferase vector and β-galactosidase vector DNA using Lipofectamine Plus reagent. Twenty-four hours after transfection, cells in duplicate plates were treated with 0, 0.1, 1 and 10 μM of BMS-345541in DMSO and incubated for an additional 36 h. Cell extract was prepared and luciferase activity was detected. Luciferase activity was normalized to β-galactosidase activity. (FIG. 2B) BMS-345541 reduction of IKK activity in SK-Mel-5 cells. 80% confluent SK-Mel-5 melanoma cells were cultured in medium containing BMS-345541 at indicated concentrations overnight (14 h). Equal aliquots of cytoplasmic IKK proteins were immunoprecipitated with both IKKα and IKKβ polyclonal antibodies and IKK activity was examined by in vitro kinase assay using substrate GST-IκBα and [γ-³²P] ATP. The [γ-³²P]-labeled IκBα protein was visualized by autoradiography. (FIG. 2C) Decrease of CXCL1 production. The supernatants from B were cleared by centrifugation, or (FIG. 2D) sera were collected from mice, and subjected to ELISA assay for CXCL1 according to the manufacture's protocol. Data shown are from a representative data set from two experiments performed in duplicate (total n=4 for each point). For ELISA; the mean value is representative of serum samples from five mice.

FIG. 3A-B. Time-lapse microscopy of NF-κB/p65 translocation and the effect of BMS-345541. SK-Mel-5 cells (2×10⁵ ) were cultured in 25 cm² culture flasks (Corning Inc., Corning, N.Y.) and transfected with 0.5 μg of GFP-p65 expression vector DNA for 14 h. Cells were treated with DMSO (sold line) or 10 μM of BMS-345541 (dashed line) one-hour prior to visualization under microscopy. The expression and localization of EGFP-p65 in live cells was recorded using time-lapse video microscopy (FIGS. 3A). The cytosolic and nuclear green fluorescent density was quantified by the Openlab software program (FIGS. 3B). Data were expressed as mean±SD (n=7 for 0 μM and n=8 for 10 μM of BMS-345541 treatment).

FIGS. 4A-4H. BMS-345541 induces apoptosis in vivo and in vitro. The cultured SK-Mel-5 cells treated with DMSO (FIG. 4A) or 10 μM of BMS-345541 (FIG. 4B) for 24 h were observed under electron microscope (arrows indicate mitochondria). Paraffin-embedded tumor tissues were subjected to H&E staining (FIGS. 4C & D) and DeadEnd Fluorometric TUNEL-assay for detection of apoptosis (FIGS. 4E & F). The TUNEL-positive cells are visualized in green fluorescence in a red (Propidium Iodide) background by fluorescence microscopy. The figure shows representative staining of tumors from vehicle treatment group (FIGS. 4C & D) and 25 mg/kg treatment group (E & F) at 20× resolutions. (FIG. 4G) Induction of apoptosis in cultured melanoma cells. 2×10⁶ SK-Mel-5 melanoma cells were cultured in medium containing the indicated concentrations of BMS-345541. After 36 h incubation, cells were collected and fixed with 1% formaldehyde and 70% ice-cold ethanol. The apoptotic cells were examined using TUNEL-assay and analyzed by flow cytometry through measuring fluorescein-12-dUTP at 520 nm. The apoptotic rate is indicated in each box. Data shown are represented one of three independent experiments. (FIG. 4H) Induction of nuclear apoptosis along with time-course. SK-Mel-5 cells were treated with 10 μM BMS-345541 for 0 h, 24 h, 38 h and 48 h. Cells were fixed and stained with Propidium iodide. Cell fluorescence was measured by flow cytometry and the data were analyzed using DNA content histogram deconvolution software (Multicycle from Phoenix Flow Systems). The percentage of apoptotic cells with fragmented DNA (PI intensity sub-G₀/G₁) is indicated at each time point. The experiments were repeated twice, in duplicates for each point (total n=4).

FIGS. 5A-5E. BMS-345541 induction of mitochondrial damage and apoptosis. (FIG. 5A) Redistribution of Bcl-2 and Bax in mitochondria. SK-Mel-5 cells were treated without or with 10 μM BMS-345541 for 24 h. Subcellular mitochondria and cytoplasm were prepared and subjected to Western blot analysis for Bcl-2 and Bax. (FIG. 5B) Dissipation of mitochondrial Δψm. SK-Mel-5 cells were exposed to 10 μM BMS-345541 for 0 h, 24 h, and 48 h. After exposure, mitochondria were isolated and incubated with 80 μM 3,3′dihexyloxacarbocyanine iodide (DiOC₆) at 37° C. for 15 min. Δψm was determined by FACS analysis using 484/530 nm filters within 10 min while gating the forward and sideward scatters on intact mitochondria (FIG. 5C). (FIG. 5D) Release of cytochrome-C and AIF. SK-Mel-5 cells were treated with or without 10 μM BMS-345541 for 24 h. Subcellular extracts were prepared and subjected to Western blot analysis for cytochrome-C and AIF. (FIG. 5E) The purified mitochondria were incubated with DMSO or 10 μM BMS-345541 at 4° C. for 30 min. The supernatant was assessed for cytochrome-c release. 200 μM Ca²⁺ was used as a positive control.

FIGS. 6A-6E. BMS-345541 induction of caspase-independent apoptosis. (FIG. 6A) Induction of caspase activity. SK-Mel-5 melanoma cells were treated for 36 h with DMSO vehicle or 10 μM BMS-345541. Pancaspase inhibitor z-VAD-fmk (50 μM) was applied one hour before BMS-345541 treatment. The cellular caspase-3 activity in samples was detected as described in Materials and Methods. (FIG. 6B) Activation of caspases slightly contributes to apoptosis. SK-Mel-5 cells were pretreated with DMSO or 50 μM Z-VAD-fmk for 1 h and then exposed to 10 μM BMS-345541 for 48 h. Cells were fixed and stained with propidium iodide staining as described in FIG. 4H. The percentage of cell death was measured with flow cytometry. (FIG. 6C) Sequence for release of mitochondrial proteins. SK-Mel-5 cells were cultured in medium containing 10 μM BMS-345541 for the indicated time. Cytosol was extracted and subjected to Western blot analysis for AIF, cytochrome-c (cyto-C) and actin. Actin was used as a loading control protein. (FIG. 6D) The inhibition of caspase failed to block AIF nuclear translocation. Western blot analysis for AIF translocation into nuclear fraction of SK-Mel-5 cells 24 h after 10 μM BMS-345541treatment in the presence of pan-caspase inhibitor (Z-VAD-fmk, 50 μM) and control cells. Ref-1 was used as a nuclear marker protein. All data shown here were from three independent experiments. (FIG. 6E) Representative confocal images for AIF translocation and nuclear condensation. SK-Mel-5 cells were exposed to 10 μM BMS and/or pan-caspase inhibitor (Z-VAD-fmk, 50 μM). The nuclear translocation of AIF (green) overlapped with nuclear PI staining (red) is noted by yellow color.

FIGS. 7A-7C. BMS-345541 induces apoptotic melanoma cells in an AIF-dependent manner. FIG. 7A, siRNA knockdown of AIF protein expression. Western blot analysis of whole cell lysate of AIF protein in control siRNA- and AIF siRNA-transfected SK-Mel-5 cells. FIG. 7B, siRNA AIF attenuation of the BMS-345541-induced nuclear apoptosis. Quantitative analysis of apoptosis in control siRNA- and AIF siRNA-transfected Sk-Mel-5 cells after treatment with 10 μM BMS-345541 for 24 h shows greater than 50% reduction in cell death in AIF siRNA transfected cells. FIG. 7C, Contribution of apoptotic proteins to the BMS-345541-induced apoptosis. Approximate percentage of apoptosis produced by the indicated apoptotic executor protein was calculated based on caspase inhibitor or siRNA knockdown application following BMS-345541 treatment.

### DESCRIPTION OF ILLUSTRATIVE EMBODIMENTS

I. The Present Invention

In order to overcome the deficiencies in the art in treating melanoma cancer, new therapeutic agents are sought which are more effective at inhibiting cancer cells, and less toxic to normal cells. Thus, the present invention provides a synthetic agent for the treatment of melanoma cancer.

NFκB and molecules in the NFκB pathway such as inhibitor of kappa-B kinase (IKK) are important targets of cancer chemotherapy. The pathogenesis of human melanoma and other cancers has been documented to be associated with constitutive activity of the kinase known as inhibitor of kappa-B kinase (IKK) (Richmond, 2002).

BMS-345541 (4(2′-aminoethyl)amino-1,8-dimethylimidazo(1,2-a)quinoxaline) was identified as a highly selective inhibitor of IKK that is able to inhibit the stimulated phosphorylation of IκBα in cells (IC₅₀=4 μM), while failing to affect 15 other kinases including JNK, or mitogen-activated protein kinase-activated protein kinase 2 (Burke et al., 2003). To determine the role of highly active IKK activity in melanoma tumorigenesis and to evaluate IKK as a therapeutic target in human melanoma, the IKK inhibitor, BMS-345541, was delivered to human melanoma cells in vitro and in vivo to examine these possibilities. It was investigated in detail the effect of BMS-345541 on IKK/NFκB or MAPK (ERK1/2) signal pathways, chemokine production, tumorigenesis and apopotosis. The results demonstrate that inhibition of constitutive IKK activation in human melanoma by BMS-345541 directly reduced NFκB activity and CXCL1 secretion, induced caspase-3 activity, and finally resulted in melanoma cellular apoptosis and antitumorigenic effects.

Thus, the present invention demonstrates that BMS-345541 has a potent antitumor effect on multidrug resistant melanoma cancer cells. Moreover, BMS-345541 induces cell-cycle arrest followed by the induction of apoptosis. These results indicate that BMS-345541 is a potent anticancer agent in melanoma cancer cells.

II. NFκB and IκB kinase in Melanoma

NPκB represents a Rel family of five proteins, c-Rel, Rel A/p65, RelB, NFκB1 (p50 and its precursor, p105), and NF-κB2/p52 and its precursor, p100 (Ghosh et al., 1998). NFκB proteins are regulated by a family of inhibitory proteins known as IκBα, IκBβ, IκBc, IκBγ or Bcl-3. In the case of NFκB1 and -2, the precursor forms p105 and p100 contain inhibitory domains which are structurally similar to the IκB proteins (Ghosh et al., 1998; Beg et al., 1992). In unstimulated cells, NF-κB is largely retained in the cytoplasm as a complex that is predominantly composed of p65 and p50 with IκBα and IκB binding to the Rel homology domain to mask their nuclear localization sequence (Baldwin, Jr., 1996; Wang et al., 1996; Huxford et al., 1998; Malek et al., 1998). The NH₂ terminus of the IκBα protein contains two serine residues that modulate signal-dependent protein stability (Maniatis, 1997) while the COOH-terminal PEST domain contributes to basal protein turnover (Rodriguez et al., 1995). IκBα as a key molecular regulator of NFκB activation is phosphorylated by IKK upon various stimuli, including cytokines such as TNFα and IL-1, or chemokines such as CXCL1 and CXCL8 (Richmond, 2002).

IκB kinase (IKK) consists of two catalytic subunits, IKKα and IKKβ, and a regulatory component, NEMO/IKKγ, which have been identified within a high molecular weight cytoplasmic complex (Mᵣ 600,000 to Mᵣ 900,000) (Mercurio et al., 1997; Woronicz et al., 1997; Regnier et al., 1997). The kinase activity of IKKα and IKKβ can be induced with cytokine challenge along with a consequent phosphorylation of IκBα at serine residues 32 and 36 (Regnier et al., 1997; Zandi et al., 1997). This is critical for subsequent ubiquitination of IκBα at lysine residues 21 and 22 by (β-transducin repeat-containing proteins (Spencer et al., 1999; Yaron et al., 1998) and degradation of IκBα by the 26S proteasome (Zandi and Karin, 1999), which leads to the exposure of nuclear localization signals on NFκB proteins which are then transported to the nucleus where they regulate NFκB responsive genes (Baldwin, Jr., 1996; Ghosh et al., 1998).

Recent studies have demonstrated that NFκB activation can maintain tumor cell viability and inhibition of NFκB alone is sufficient to induce apoptosis (Mathas et al., 2003; Cahir-McFarland et al., 2000; Keller et al., 2000). Previous studies also provided evidence that IKK is constitutively active in human melanoma cells, which leads to NFκB activation and results in aberrant overexpression of chemokines such as CXCL1 and CXCL8 (Yang and Richmond, 2001). This has been implicated in transformation and melanoma tumor progression both in vivo and ex vivo (Richmond, 2002, Haghnegahdar et al., 2000; Liptay et al., 2003). It has also been demonstrated that the CXCL1 chemokine has potency in induction of IKK activation in normal human melanocytes (Yang and Richmond, 2001) and potentiates melanoma formation in a mouse model deficient for inhibitor of kinase 4a (INK4a)/alternate reading frame (ARF) (Yang et al., 2001). In agreement with these observations and other reports, highly activated members of the NFκB family were found to be responsible for the development of melanoma and other cancers (Yang and Richmond, 2001; Shattuck-Brandt and Richmond, 1997; Sylla and Temin, 1986; Moore and Bose, 1996; Gilmore et al., 1996; White et al., 1996). Since IKK is a key molecular complex specifically regulating IκB proteins and subsequently targeting NFκB, it was hypothesized that IKK would be a good therapeutic target for malignant melanoma.

III. BMS-345541 Compounds as Therapeutic Agents

The present invention relates to (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid (BMS-345541; Burke et al., 2003), or salt, or an analog thereof that demonstrate potent anti-cancer activity in melanoma cancer cells.

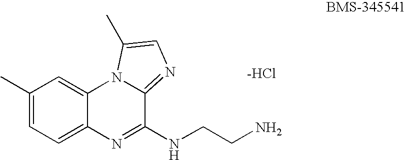

BMS-345541 may be prepared by methods as disclosed by Burke et al., 2003; and U.S. Patent Publication 20030022898 each incorporated herein by reference. See also U.S. Pat. Nos. 6,235,740; 6,239,133; and 6,635,626.

Synthesis of BMS-345541 (4(2′-Aminoethyl)amino-1,8-dimethylimidazo(1,2-a)quinoxaline)—

4,5-Dihydro-1,8-dimethylimidazo(1,2-a)quinoxalin-4-one-2-carboxylic acid, was suspended in diphenyl ether (124 mmol in 400 ml) and refluxed at 260° C. for 2 h. After the suspension cooled down to room temperature, hexanes (C₆H₁₄; 500 ml) were added to further precipitate the product. The solid was filtered, giving 4,5-dihydro-1,8-dimethylimidazo(1,2-a)quinoxalin-4-one as a white solid (29.6 g). ¹H NMR (500 MHz; hexadeuterio-dimethyl sulfoxide, d₆-Me₂SO) δ 11.7(s, 1H), 7.91 (s, 1H), 7.31 (s, 1H), 7.29 (d, J=8.1 Hz, 1H), 7.22 (d, J=8.3 Hz, 1H), 2.81 (s, 3H), 2.41 (s, 3H); MS (electrospray ionization), m/z 214.13 ((M+H)⁺; calculated for C₁₂H₁₁N₃O, 213.09). A mixture of 4,5-dihydro-1,8-dimethylimidazo(1,2-a)quinoxalin-4-one (29.6 g, 139 mmol) and N,N-diethylaniline (PhNEt₂; 45 ml) was refluxed in phosphorus oxychloride (POCl₃; 250 ml) for 1 h. The solvent was evaporated under vacuum; the residue was diluted with CHCl₃ (1000 ml), followed by careful neutralization with cold saturated Na₂CO₃ solution. The aqueous layer was further extracted with CHCl₃. The combined organic layer was dried over MgSO₄, filtered, and concentrated. Flash chromatography using ethyl acetate and hexanes (EtOAc/hexanes, 60/40) provided 4-chloro-1,8-dimethylimidazo(1,2-a)quinoxaline as a white solid (25 g). ¹H NMR (500 MHz; CDCl₃) δ 8.07 (s, 1H), 7.93 (d, J=8.3 Hz, 2H), 7.54 (d, J=0.7 Hz, 1H), 7.41 (dd, J=8.4, 1.2 Hz, 1H), 2.97 (s, 3H), 2.59 (s, 3H); MS (electrospray ionization), m/z 232.04 ((M+H)⁺; calculated for C₁₂H₁₀ClN₃, 231.06). A solution of 4-chloro-1,8-dimethylimidazo(1,2-a)quinoxaline (4.8 g, 21 mmol) in ethylenediamine (300 ml) was heated at 60° C. under nitrogen gas (N₂) for 16 h. The solvent was evaporated under vacuum. The residue was diluted with EtOAc (200 ml), washed with saturated Na₂CO₃ and brine, dried over MgSO₄, filtered, and concentrated. Flash chromatography using methanol (MeOH) provided a solid, which was then dissolved with CHCl₃ and filtered to remove any dissolved silica gel. The filtrate was concentrated under vacuum, providing 4(2′-aminoethyl)amino-1,8-dimethylimidazo(1,2-a)quinoxaline as a white solid (4.67 g). ¹H NMR (500 MHz, CDCl₃) δ 7.88 (s, 1H), 7.63 (d, J=8.3 Hz, 1 H), 7.26 (s, 1 H), 7.22 (d, J=8.3 Hz, 1H), 6.31 (s, 1H), 3.63 (apparent q, J=5.9 Hz, 2H), 2.96 (t, J=6.0 Hz, 2H), 2.88 (s, 3H), 1.38 (br, 2H); MS (electrospray ionization), m/z 256.15 ((M+H)⁺; calculated for C₁₄H₁₇N₅, 255.15). 4(2-aminoethyl)amino-1,8-dimethylimidazo(1,2-a)quinoxaline (4.67 g, 8.3 mmol) was dissolved with aqueous hydrochloric acid, HCl (1.0 N, 18.3 ml), and water, H₂O (50 ml), at room temperature. Subsequent removal of water using a lyophilizer provided the hydrochloride salt as a white solid (5.22 g).

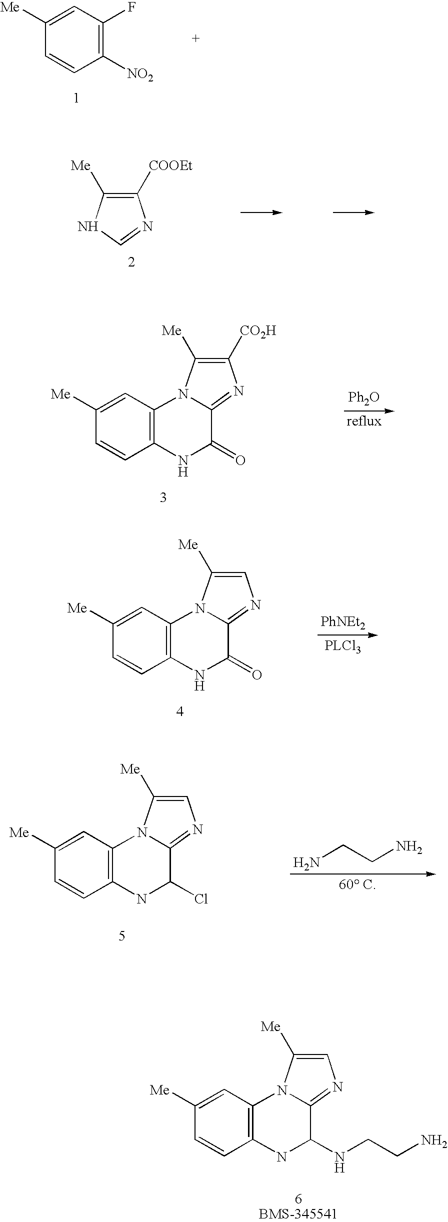

BMS-345541 is shown to inhibit cell growth and is therefore useful in the treatment of diseases of uncontrolled proliferation, such as cancer. Thus, the present invention provides BMS-345541 or an analog thereof as a therapeutic agent for treating melanoma cancer in a subject.

In some instances, the cancer to be treated using BMS-345541 or an analog thereof may be a multidrug-resistant cancer such as, but not limited to, a paclitaxel-resistant cancer.

To kill cells, induce cell cycle arrest, inhibit cell growth, inhibit metastasis, inhibit angiogenesis or otherwise reverse or reduce the malignant phenotype of melanoma cancer cells, using the methods and compositions of the present invention, one would generally contact a cell with the BMS-345541 compound, or salts or analogs thereof. The terms “contacted” and “exposed,” when applied to a cell, are used herein to describe the process by which a therapeutic agent is delivered to a target cell or are placed in direct juxtaposition with the target cell. To achieve cell killing or stasis, the therapeutic agent is delivered to a cell in an amount effective to induce cell cycle arrest, inhibit cell growth and induce apoptosis in the cell.

BMS-345541 or a salt or an analog thereof as a therapeutic agent may be administered to a subject more than once and at intervals ranging from minutes to weeks. One would generally ensure that a significant period of time did not expire between the time of each delivery, such that the agent would still be able to exert an advantageous effect on the cell. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of BMS-345541 or a salt or an analog thereof to a subject may be by any method know in the art for delivery of a therapeutic agent to a subject. For example, such methods may include, but are not limited to, oral, nasal, intramuscular, or intraperitoneal administration. Methods of administration are disclosed in detail elsewhere in this application.

IV. Assaying for the Effect of BMS-345541 Compound in Melanoma Cancer Cells

The present invention comprises methods for assaying for the inhibition of melanoma cancer cell growth comprising:

a) providing a melanoma cancer cell sample;

b) contacting the cell sample with an effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or a salt, or an analog thereof;

c) analyzing the cell sample for growth inhibition; and,

d) comparing the inhibition of the cell growth in step (c) with the inhibition of a melanoma cancer cell in the absence of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or a salt, or an analog thereof, wherein the difference in growth inhibition represents the growth inhibitory effect of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid, or a salt, or an analog thereof.

BMS-345541 may be used to induce cell cycle arrest, inhibit cell growth or induce apoptosis in cells such as a melanoma cancer cell. Assays employed by the present invention to observed these effects of BMS-345541 on a cancer cell are well known to one of ordinary skill in the art and may include, but are not limited to FACS analysis or TUNEL assay, and caspase assays.

1. FACS Analysis

Fluorescent activated cell sorting, flow cytometry or flow microfluorometry provides the means of scanning individual cells to identify those cells in the population of cells that are apoptotic. The method employs instrumentation that is capable of activating, and detecting the excitation emissions of labeled cells in a liquid medium.

FACS is unique in its ability to provide a rapid, reliable, quantitative, and multiparameter analysis on cells.

2. TUNEL Assay

The present invention may also employ the use of the TUNEL assay in examining the effects of BMS-34551 or an analog thereof on melanoma cancer cells. Various procedures for TUNEL assay are well known to those of ordinary skill in the art. For example, in the present invention, DeadEnd™ Fluorimetric TUNEL System from Promega Corporation (Madison, Wis.) was used to detect apoptosis in tumor tissue embedded in paraffin. This assay may be performed following the manufacturer's protocol. Briefly, tissue sections are deparaffinized in Xylene and rehydrated in graded ethanol washes. The tissue sections are then fixed with methanol-free paraformaldehyde and treated with 20 μg/ml Proteinase K. The sections are then washed and incubated inside a humidified chamber with TdT incubation buffer containing equilibration buffer, nucleotide mix and TdT enzyme, for 60 minutes at 37° C. The reaction is terminated by immersing the sections in 2×SSC. After staining sections with 500 ng/ml propidium iodide, the samples are immediately analyzed under a fluorescence microscope using a standard fluorescein filter set to view the green fluorescein at 520 nm and red fluorescence of propidium iodide at >620 nm. The slides are imaged with 10× and 20× objectives using an Openlab™ -controlled microscope.

3. Annexin Assay

The present invention may also employ the use of the Annexin V assay in examining the effects of BMS-34551 or an analog thereof on melanoma cancer cells. An early process in apoptosis is the flipping of some components of the plasma membrane from the inside surface to the outside surface, principally phosphatidylserine (PS). “Flipping” is the transition from a normally asymmetric distribution of the charged head groups of PS to a random orientation, thus exposing the PS head group on the external surface of the plasma membrane. Annexin V, a phospholipid binding protein with the convenient habit of binding to PS provides an identification tag for apoptotic cells. Annexin V conjugated with a variety of labels and in kits of various kinds is available for identifying apoptotic cells. In addition to the standard fluoroscein isothiocyanate (FITC), biotin, and phycoerythrin (PE), Annexin is also available with allophycocyanin (APC) (ALEXIS), Alexa™ 488 (Molecular Probes, NeXins Research), Oregon Green™ (Molecular Probes, NeXins Research, R & D Systems), Alexa 568 (Molecular Probes, NeXins Research, Roche Molecular Biochemicals), and enhanced green fluorescent protein (CLONTECH).

Annexin V labeled cells can be assayed either by flow cytometry, when a fluorochrome is attached to Annexin, or on slides. Both techniques give information on the percentage of cells in a population undergoing apoptosis and, in the case of cytometry, can be used to isolate such cells.

4. DNA Fragmentation Assay

Another approach that may be employed in the present invention to detect the effect of BMS345541 or an analog thereof on melanoma cancer cells may take advantage of the fact that during apoptosis, DNA content will slowly change as proteins are stripped off the chromosomes and the DNA becomes vulnerable to nicking by endonucleases. In permeabilized cells, low molecular weight DNA will leak out of the nucleus, leaving cells with a lowered DNA content, which can be documented with DNA stains.

The fragmentation of the nuclear DNA and concomitant generation of free 3′-ends may be assessed by TUNEL (terminal deoxynucleotidyl transferase mediated dUTP nick end labeling) assays, which enzymatically incorporate modified nucleotides containing a variety of tags on the ends, marking apoptotic cells for flow cytometry or microscopy. TUNEL kits are well known in the art

In addition, the labeling of apoptotic cells with a monoclonal antibody directed against single-stranded DNA, offered by APOSTAIN and ALEXIS, provides another method for assaying apoptosis-induced DNA fragmentation, with respect to specificity (Frankfurt et al., 1996). This stain is based on the increased sensitivity of apoptotic cells to heat; gentle heating unravels the DNA in apoptotic cells due to the loss of chromosomal protein. The antibody binds to this unraveled single-stranded DNA.

5. Other Apoptotic Assays

The effect of BMS-345541 or an analog thereof on the apoptosis of melanoma cancer cells may be assessed using antibodies directed to the apoptotic pathway such as caspase-3, and PARP but are not limited to such. A number of purified enzymes of caspases, antibodies directed against caspases, substrates and inhibitors of the enzymes, and kits for assaying caspases, are available in the art. In addition, reagents that will detect the cleaved protein products of various caspases are well know to one of ordinary skill in the art.

Additionally, a variety of polyclonal antibodies directed against other apoptosis targets (for example PARP, DFF) are available from a number of companies (Pharmingen, BIOMOL, Enzyme Systems Product, Affinity BioReagents, and others). Most, but not all, of these recognize both cleaved and native species and therefore must be used with separated proteins, on Westerns, for example. However, antibodies that recognize only the cleaved and hence activated caspase 3 are available from Pharmingen and Upstate Biotechnology. Promega also provides an anti-PARP p85 fragment polyclonal antibody that will specifically recognize the caspase cleavage fragment of PARP without recognizing the 116 k Da intact molecule in formalin fixed samples for immunocytochemistry.

Western blotting (immunoblotting) analysis is well known to those of skill in the art, see U.S. Pat. No. 4,452,901 incorporated herein by reference and Sambrook et al. (2001). In brief, this technique generally comprises separating proteins in a sample such as a cell or tissue sample by SDS-PAGE gel electrophoresis. In SDS-PAGE proteins are separated on the basis of molecular weight, then are transferring to a suitable solid support, (such as a nitrocellulose filter, a nylon filter, or derivatized nylon filter), followed by incubation of the proteins on the solid support with antibodies that specifically bind to the proteins.

Caspase 3 may also be assayed in melanoma lysates using the Fluorescence Immunoabsorbent Enzyme Assay (FIENA) Kit (Roche Molecular Biochemicals). This kit incorporates an adsorption step on microplates coated with anticaspase 3 antibody, and is provides more specificity than using the substrates alone.

6. Cell Count Assay

Apoptosis may also be observed by the uptake of vital dyes, fluorescent (propidium iodide or PI) or colorimetric (trypan blue). The effect of BMS-3435541 or an analog thereof on melanoma cancer cells may then by determined by microscopy or by FACS analysis by counting the number of apoptotic cells in the population of cells.

V. Combined Cancer Therapy

In the context of the present invention, it is contemplated that the BMS-345541 compound, or salts or analogs thereof may be used in combination with a second therapeutic agent to more effectively treat a melanoma cancer. Additional therapeutic agents contemplated for use in combination with the BMS-345541 compound, or salts or analogs thereof include, but are not limited to anticancer agents. Anticancer agents may include but are not limited to, radiotherapy, chemotherapy, gene therapy, hormonal therapy or immunotherapy that targets cancer/tumor cells.

To kill cells, induce cell-cycle arrest, inhibit cell growth, inhibit metastasis, inhibit angiogenesis or otherwise reverse or reduce the malignant phenotype of melanoma cancer cells, using the methods and compositions of the present invention, one would generally contact a cell with a BMS-345541 compound, or salts or analogs thereof in combination with a second therapeutic agent. These compositions would be provided in a combined amount effective to kill or inhibit proliferation of the cell. This process may involve contacting the cells with BMS-345541, or salts or analogs thereof in combination with a second therapeutic agent or factor(s) at the same time. This may be achieved by contacting the cell with a single composition or pharmacological formulation that includes both agents, or by contacting the cell with two distinct compositions or formulations, at the same time, wherein one composition includes the BMS-345541 or derivatives thereof and the other includes the second agent.

Alternatively, treatment with BMS-345541, or salts or analogs thereof may precede or follow the additional agent treatment by intervals ranging from minutes to weeks. In embodiments where the second agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either BMS-345541 or salts, or analogs thereof in combination with a second therapeutic agent such as a anticancer agent or anticancer agent will be desired. Various combinations may be employed, where BMS-345541 or salts or analogs thereof is “A” and the second therapeutic agent is “B”, as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B
Other combinations are contemplated. Again, to achieve cell killing by the induction of apoptosis, both agents may be delivered to a cell in a combined amount effective to kill the cell.

A. Chemotherapeutic Agents

The present invention also contemplates the use of chemotherapeutic agents in combination with BMS-345541 or an analog thereof in the treatment of melanoma cancer. Examples of such chemotherapeutic agents may include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, gemcitabien, navelbine, farnesyl-protein transferase inhibitors, transplatinum, 5-fluorouracil and methotrexate, or any analog or derivative variant of the foregoing.

B. Radiotherapeutic Agents

Radiotherapeutic agents may also be use in combination with the compounds of the present invention in treating a melanoma cancer. Such factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapeutic Agents

Immunotherapeutics may also be employed in the present invention in combination with BMS-345541 or salts or analogs thereof in treating melanoma cancer. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene. and are essential or the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1; NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

E. Regulators of Programmed Cell Death

Apoptosis, or programmed cell death, is an essential process in cancer therapy (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Members of the Bcl-2 that function to promote cell death such as, Bax, Bak, Bik, Bim, Bid, Bad and Harakiri, are contemplated for use in combination with BMS-345541 or an analog thereof in treating melanoma cancer.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas /Fas ligand, DR4 or DR5/TRAIL would potentiate the apoptotic inducing abilities of the present invention by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

G. Surgery

It is further contemplated that a surgical procedure may be employed in the present invention. Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with a second anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

VI. Formulations and Routes for Administration

Where clinical applications are contemplated, it will be necessary to prepare pharmaceutical compositions of BMS-345541 or salts or analogs thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, nasally, intramuscularly, intratumorally, intraperitoneally. In some embodiments, local or regional delivery of BMS-345541 or salts or analogs thereof alone, or in combination with a second therapeutic agent, to a patient with cancer or pre-cancer conditions will be a very efficient method of delivery to counteract the clinical disease. Similarly, chemo- or radiotherapy may be directed to a particular, affected region of the subject's body. Regional chemotherapy typically involves targeting anticancer agents to the region of the body where the cancer cells or tumor are located. Other examples of delivery of the compounds of the present invention that may be employed include intra-arterial, intracavity, intravesical, intrathecal, intrapleural, and intraperitoneal routes.

Intra-arterial administration is achieved using a catheter that is inserted into an artery to an organ or to an extremity. Typically, a pump is attached to the catcher. Intracavity administration describes when chemotherapeutic drugs are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system.

Alternatively, systemic delivery of the chemotherapeutic drugs may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. This includes but is not limited to, oral, nasal, or buccal routes. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant , such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with BMS-345541 or salts or analogs thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

VII. Therapeutically Effective Amounts of BMS-345541 Compositions

A therapeutically effective amount of BMS-345541, or salts, or analogs thereof alone, or in combination with a second therapeutic agent such as an anticancer agent as a treatment varies depending upon the host treated and the particular mode of administration. In one embodiment of the invention the dose range of the BMS-345541 or salts or analogs thereof alone, or in combination with a second agent used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for BMS-345541, or salts or analogs thereof in combination with a second therapeutic agent.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as BMS-345541 or salts or analogs thereof alone, or in combination with a second therapeutic agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, BMS-345541 or salts or analogs thereof alone, or in combination with a second therapeutic agent will be administered. When a second therapeutic agent is administered, as long as the dose of the second therapeutic agent does not exceed previously quoted toxicity levels, the effective amounts of the second therapeutic agents may simply be defined as those amounts effective to reduce the cancer growth when administered to an animal in combination with the BMS-345541 or salts or analogs thereof. This may be easily determined by monitoring the animal or patient and measuring those physical and biochemical parameters of health and disease that are indicative of the success of a given treatment. Such methods are routine in animal testing and clinical practice.

In some embodiments of the present invention chemotherapy may be administered, as is typical, in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents and Cell Culture.

BMS-345541 (4(2′-aminoethyl)amino-1,8-dimethylimidazo(1,2-a)quinoxaline)-4,5-Dihydro-1,8-dimethylimidazo(1,2-a)quinoxalin-4-one-2-carboxylic acid was prepared by the described procedure (Burke et al., 2003) in Bristol-Myers Squibb Pharmaceutical Research Institute. BMS-345541 was dissolved in DMSO to make up 50 mM stock solution for in vitro experiments or stock solutions of BMS-345541 (10, 25 and 75 mg/10 ml) were dissolved in water with addition of equal moles of hydrogen chloride (pH 7.0) for in vivo experiments. A super-repressor form of human IκBα (S32, 36A) resistant to degradation and mutant IKKβ (K44M) was kindly provided by Javier Piedrafita (Sidney Kimmel Cancer Center, University of California-San Diego School of Medicine). Antibodies to IKKα (H-744), IKKβ (H-470), Bcl-2, Bax and AIF were purchased from Santa Cruz Biotechnology, Inc. (Santa Cruz, Calif.). Dihydroethidine, 3,3′dihexyloxacarbocyanine iodide and pan-caspase inhibitor (Z-VAD-FMK) were purchased from Molecular Probes (Eugene, Oreg.). N-acetyl-L-cysteine (NAC) and Tiron were from Sigma-Aldrich (St. Louis, Mo.). Normal human epidermal melanocytes (NHEM) were provided by the Skin Disease Research Center in Vanderbilt University School of Medicine. NHEM were cultured in 154 medium with 1× human melanocyte growth supplement (Cascade Biologicals, Inc.). The melanoma cell lines, Sk Mel5, A375 and Hs294T, originally established from human metastatic melanoma, were purchased from American Type Culture Collection and cultured in DMEM: Ham's F-12 medium containing 10% FBS, 2 mM of L-glutamine, 100 μM of MEM Non-Essential Amino Acids (Life Technologies, Inc.), and 1 mM of sodium pyruvate (Sigma Chemical Co.).

Approaches of Drug Delivery and Tumor Measurement.

Animal experimentation was undertaken according to protocols approved by the Institutional of Animal Care and Use Committee at Vanderbilt University. BMS-345541 solution at 10 ml per kg body weight was orally administered to the mouse using a modified dull 19G1_{1/2}-gauge needle connected to a 1 ml syringe. Tumor size was measured with an electronic digital caliper. Tumor volume was calculated by width²×length×0.52 and expressed as mean±SD mm³.

Immunoprecipitation and Kinase Assay in Vitro.

To analyze the IKK activity, cytoplasmic extracts were prepared from cells cultured for 24 h in serum-free medium. Cells were mechanically released from tissue culture plates by scraping in cold PBS according to standard protocols. Cells were collected by centrifugation (800×g), and then resuspended in lysis buffer [10 mM HEPES (pH 7.9), 10 mM KCl, 0.1 mM EDTA, 0.1 mM EGTA, 0.1% NP40, 1 mM DTT, and 0.5 mM phenylmethylsulfonyl fluoride] with Complete Protein Inhibitors (Roche). 400 μg aliquots of cytoplasmic extracts were incubated with 1 μg each of IKKα and IKKβ polyclonal antibodies (Santa Cruz Biotechnology) and 60 μg aliquots of Protein A/G agarose-conjugated beads for 4 h at 4° C. After washing twice with wash buffer C [50 mM HEPES (pH 7.0), 250 mM NaCl, 5 mM EDTA, and 0.1% NP40] and once with kinase buffer [20 mM HEPES (pH 7.4), 10 mM MgCl₂, 2 mM MnCl₂, 25 mM β-glycerophosphate (Sigma Chemical Co. G-6251), 4 mM NaF, 0.1 mM sodium orthovanadate, and 1 mM DTT], the beads were mixed with 20 μl of kinase buffer containing 100 μM ATP, 5 μCi of [γ⁻³²P] ATP, and 1 μg of glutathione S-transferase (GST) fused protein of IκBα (amino acids 1-54) as a substrate of the IκB kinase and incubated at 30° C. for 30 min. The reaction mixtures were resolved by SDS-PAGE, transferred to nitrocellulose membrane, and phosphorylated IκB was detected by autoradiography.

Western Blot Analysis.

Proteins from cell extracts were separated by 12% SDS-PAGE and electrophoretically transferred to nitrocellulose membranes. Nonspecific binding was blocked with 5% milk in TBST buffer (4 mM Tris base, 100 mM NaCl, pH 7.5, 0.05% Tween), incubated with the indicated primary antibodies with a dilution of 1:1000 at room temperature for 2 h or 4° C. for overnight and then with horseradish peroxidase-conjugated secondary antibody for 1 h. Blots were visualized by enhanced chemiluminescence assay.

Transfection and Luciferase Reporter Activity Assay.

Melanoma cells were seeded in six-well plates and the next day 80% confluent cells were transfected with the respective constructs using FuGENE 6 Transfection Reagent (Roche Diagnostics Corporation, Indianapolis, Ind.) following the manufacturer's protocol. The HIV-long terminal repeat-luciferase construct, containing two NF-κB binding sites, was the kind gift of Dr. Richard Gaynor (University of Texas Southwest Medical Center, Dallas, Tex.). The pRSV β-galactosidase reporter vector was purchased from Promega Corp. (Madison, Wis.). Extracts from cultured cells were prepared, and luciferase and β-galactosidase activities were detected using a Dual-Light kit (Tropix) according to the manufacturer's instruction.

ELISA Assay.

To quantify the CXCL1 expression levels in vivo and in vitro, serum was collected at the end of the experiments from mice bearing SK-Mel-5 melanoma tumors or from the cleared supernatants of melanoma cell culture medium. Briefly, 1.5×10⁵ of SK-Mel-5 cells/well were seeded in six-well plates in duplicate in DMEM/F12 medium containing 10% FBS. After overnight incubation, the cell monolayers were washed with serum-free culture medium and then incubated with the different concentrations of BMS-345541 for an additional 14 h at 37° C., after which the supernatant was collected and cleared by centrifugation. Aliquots were then subjected to CXCL1 ELISA assay (R&D system) according to the manufacturer's protocol.

Caspase-3 Activity Assay.

The activation of caspase-3 was detected with the Caspase-3 Cellular Activity Assay Kit PLUS obtained from Biomol (Plymouth Meeting, Pa.), which uses N-acetyl-Asp-Glu-Val-Asp-p nitroaniline (Ac-DEVD-pNA) as a substrate. In brief, 10 μg of cytosol protein extract prepared in lysis buffer (50 mM HEPES, pH 7.4, 0.1% CHAPS [3-[(3-cholamidopropyl)-dimethylammonio]-1-propanesulfonate], 5 mM DTT, 0.1 mM EDTA and 0.1% tween 20) was diluted to a total volume of 100 μl of caspase buffer (50 mM HEPES, pH 7.4, 100 mM NaCl, 0.1% CHAPS, 10 mM DTT, 1 mM EDTA and 10% glycerol) containing 200 μM Ac-DEVA-pNA substrate. The mixture was incubated at 37° C. for 10 min and the released p-NA was measured at a test wavelength of 405 nm and a reference wavelength of 595 nm. Simultaneously, 15 units of human recombinant caspase-3 enzyme was assayed as a positive control, and purified caspase-3 or cytosol sample extract were pre-treated with 0.1 μM inhibitor (Ac-DEVD-CHO) as an inhibitor-treated control. 50 μM of p-NA standard was measured for calibration of the specific caspase-3 activity in samples. Experiments were repeated twice and duplicate samples were analyzed for each experiment.

Preparation of Subcellular Fractions.

The Mitochondria Isolation Kit (Pierce, Rockford, Ill.) was used for isolation of mitochondria from cultured melanoma cells according to the manufacture's instruction for the Reagent-based method. Briefly, cells (2×10⁷) were collected, washed with PBS buffer and resuspended in 0.8 ml of Reagent A on ice for 2 min. 10 μl of Reagent B was added and samples were kept on ice, with vortexing very minute for 5 min. After addition of 0.8 ml of Reagent C, the mixture was centrifuged at 700×g for 10 min to remove the cell debris and nuclei. The supernatant was then centrifuged at 3,000×g for 15 min. The pellet (mitochondria) was washed with Reagent C. The purity of mitochondria was determined by Western blotting with β-actin, where β-actin negative preparation were fully to be pure. The supernatant (cytosolic fraction) was further cleared by centrifugation at 12,000×g for 15 min. The whole cell lysate and nuclear preparation procedures previously described (Yang & Richmond, 2001).

Flow Cytometry.

For quantification of cell death, DNA content was analyzed on a FACScan (Becton Dickinson, San Jose, Calif.). Briefly, SK-Mel-5 cells were treated with BMS-345541 at different concentrations or for different time courses. The cells were collected by trypsinization, fixed in 70% ethanol for 2 h on ice and stained with propidium iodide (PI) solution (phosphate-buffered saline containing 2 μg/ml PI, 0.1% Triton X-100 and 125 units/ml RNase A) at 37° C. for 30 min. Cell fluorescence was measured by flow cytometry with 488 nm excitation and 620 nm emission filters and data were analyzed using DNA content histogram deconvolution software (Multicycle, Phoenix Flow Systems). To determine mitochondria membrane potential (Δψm), the purified mitochondria were resuspended in buffer (225 mM mannitol, 75 mM sucrose, 10 mM KCl, 10 mM Tris-HCl, 5 mM KH₂PO₄, pH 7.2) and incubated with 80 nM DiOC6 (3) for 15 min at 37° C., followed by FACS analysis with 488 nm excitation and 530 emission filters. To evaluate the generation of mitochondrial reactive oxygen species (ROS), cells treated or not treated with BMS-345541 were incubated in 5 μM dihydroethidine at 37° C. for 15 min, harvested by trypsinization, and washed with cold PBS solution three times. ROS were determined by FACS analysis with 488 nm excitation and 585 emission filters.

Microscopy.

For electron microscopy (EM), SK-Mel-5 cells treated with BMS-345541 were pelleted by centrifugation and fixed for 1 h in cold glutaraldehyde (2%) in cacodylate buffer and postfixed for 1 h with OsO₄. The pellet was embedded in epoxy resin, sectioned and collected on nickel grids. The thin sections were stained with uranyl acetate and lead citrate for respective 10 min and 15 min for EM observation. For confocal microscopy, SK-Mel-5 cells treated with BMS-34541 were fixed in 4% paraformaldehyde, blocked with 2% bovine serum albumin in PBS containing 0.5% Triton X-100. Cells were incubated with the anti-AIF polyclonal antibody for 2 h. Cells were washed twice and incubated with the secondary antibody conjugated with FITC for 1 h. Nuclei were stained with 0.5 μg/ml propidium iodide for 10 min. After washing three times with PBS, coverslips were mounted onto microscope slides. The slides were analyzed using a confocal laser-scanning microscope.

TUNEL Assay.

The DeadEnd™ Fluorimetric TUNEL System from Promega Corporation (Madison, Wis.) was used to detect apoptosis in tumor tissue embedded in paraffin. The assay was performed following the manufacturer's protocol. Briefly, tissue sections were deparaffinized in xylene and rehydrated in graded ethanol washes. The tissue sections were then fixed with methanol-free paraformaldehyde and treated with 20 μg/ml Proteinase K. The sections were then washed and incubated inside a humidified chamber with TdT incubation buffer, containing equilibration buffer, nucleotide mix and TdT enzyme, for 60 min at 37° C. The reaction was terminated by immersing the sections in 2×SSC. After staining sections with 500 ng/ml propidium iodide, the samples were immediately analyzed under a fluorescence microscope using a standard fluorescent filter set to view the green fluorescence at 520 nm and red fluorescence of propidium iodide at >620 nm. The slides were imaged with 10× and 20× objectives using an Openlab™ controlled microscope.

Small Interfering RNA Transfection.

RNA interference of AIF was performed using siGENOME™ SMARTpool® siRNA (Dharmacon Inc., Chicago, Ill.). A nonrelated siRNA that targeted the green fluorescent protein was used as a control. For transfection, SK-Mel-5 cells at 50% confluence were seeded in six-well plates and transfected with 100 nM siRNA using FuGENE 6 Transfection Reagent (Roche Diagnostics Corporation, Indianapolis, Ind.) according to manufacturer's recommendations. The protein level of AIF was detected in the whole cell lysate by Western blotting. Three successive rounds of transfection were performed until over 90% of AIF protein was knocked down. Each transfection lasted for 24 h in 10% FBS medium along with an interval of 12 h between transfections to allow time to recover cells from the toxic transfection reagent.

Statistical Analysis.

Results are expressed as mean±S.D. from three independent experiments or representative replicate experiments. Statistical analysis was performed using the unpaired Student's t test. The value of p<0.05 was considered statistically significant.

### Example 2

### Results

BMS-345541 Inhibits Growth of Melanoma Cells in Vitro and Melanoma Tumors in Vivo.

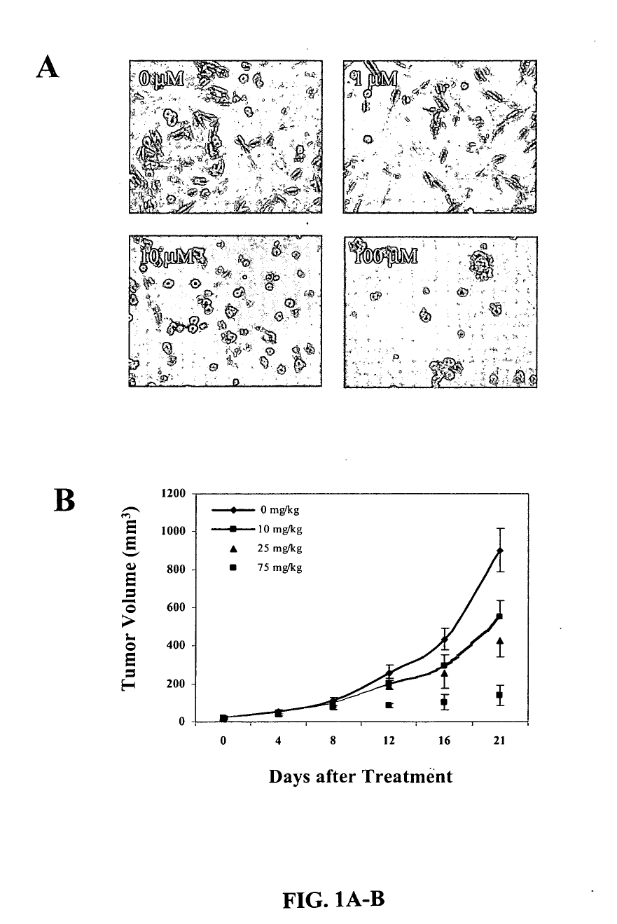

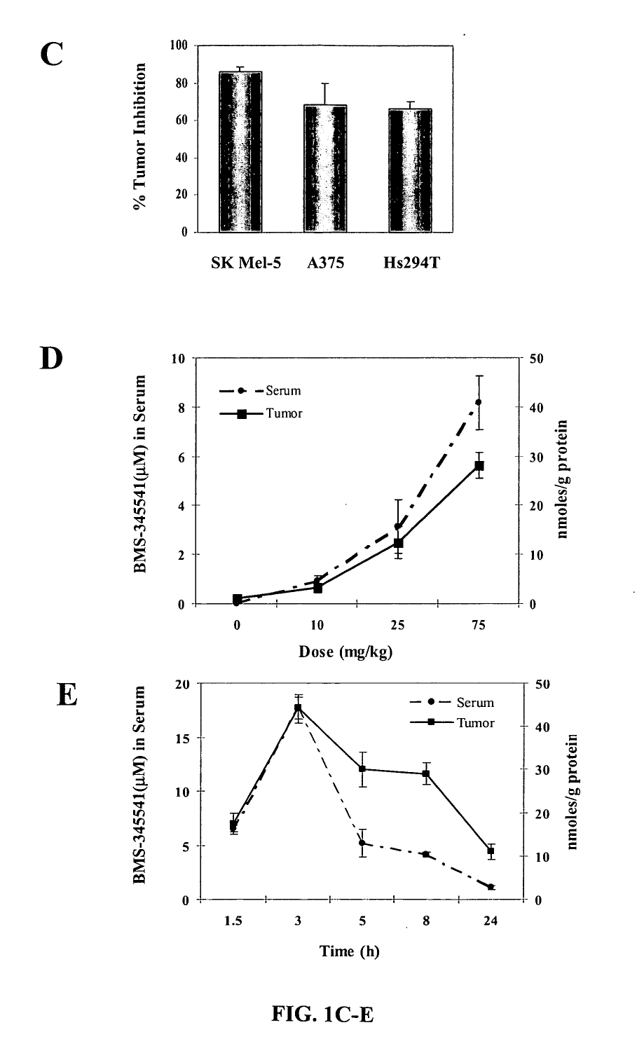

Aberrant activation of NF-κB has been associated with carcinogenesis, and constitutively high IKK activity has been detected in many tumor types, including human melanoma (Yang & Richmond, 2001; Liptay et al., 2003; Mathas et al., 2003). IKK is a major regulator of the NF-κB pathway and therefore represents an attractive therapeutic target in advanced cancers. In the present study, the inventors examined the effect of BMS-345541 as a highly selective IKKβ inhibitor on melanoma tumorigenesis. The SK-Mel-5 cell line used in this study was established from human metastatic melanoma and it exhibits high constitutive IKK activity and CXCL1 secretion (Yang & Richmond, 2001). Melanoma cells (1×10⁵ cells per well) of SK-Mel-5, A375 and Hs 294T cell lines were cultured in medium with BMS-345541 at 0, 0.1, 1.0, and 10 μM concentrations. After three days of culture, the cell numbers were determined by hemocytometer counting (Table 1). These data suggest that BMS-345541 treatment resulted in a concentration-dependent inhibition of melanoma cell proliferation. Cells treated for 14 h with 10 μM or 100 μM of BMS-345541 appeared morphologically apoptotic (FIG. 1A).

TABLE 1
BMS-34554 Inhibits Growth of the Cultured Melanoma Cells

| BMS- | | | | |
|---|---|---|---|---|
| 345541 | | Sk-Mel-5 | A375 | Hs 294T |
| (μM) | NHEM | (10⁵) | (10⁵) | (10⁵) |
| 0 | 4.8 ± 0.23 | 4.0 ± 0.15 | 3.9 ± 0.32 | 2.0 ± 0.20 |
| 0.1 | 4.8 ± 0.28 | 3.7 ± 0.55 | 3.2 ± 0.38 | 1.9 ± 0.15 |
| 1.0 | 4.9 ± 0.38 | 2.7 ± 0.31 | 1.6 ± 0.38 | 2.2 ± 0.42 |
| 10 | 0.18 ± 0.04 | 0.028 ± 0.02 | 0.020 ± 0.01 | 0.013 ± 0.01 |
| 100 | | 0 | 0 | 0 |

To further evaluate the in vivo specificity of induction of melanoma death by BMS-345541, SK-Mel-5, A375 and Hs 294T melanoma cells (2×10⁶) were subcutaneously inoculated into nude mice and melanoma tumor formation was followed. When the tumor volume reached approximately 20 mm³, BMS-345541 (0, 10, 25 and 75 mg/kg body weight) was administered to mice orally once daily for the duration of 21 days. FIG. 1B demonstrates that BMS-345541 effectively inhibits SK-Mel-5 tumor growth in a dose-dependent manner. Tumor-bearing mice treated with 75 mg/kg of BMS-345541 showed that effective inhibition of growth of SK-Mel-5, A375 and Hs 294T tumors by 86±2.8%, 69±11% and 67±3.4%, respectively, when compared with control animals treated with vehicle alone (FIG. 1C). BMS-345541 treatment was well tolerated by the mice. However, loss of body weight was observed during the therapeutic process. An average 3%, 5%, 9% and 14% loss of body weight was observed in mice receiving 0, 10, 25 and 75 mg/kg BMS-345541, respectively. Tumor-bearing mice were orally treated with daily 60 mg/kg dosage of the DNA alkylating agent, temozolomide (Spiro et al., 2000; Frick et al., 2002) and showed tumor growth inhibition by 81%. These data demonstrate that BMS-345541 is reasonably well tolerated by the mice, even at high dose and is approximately as effective as high doses of temzolomide.

Since BMS-345541 growth-inhibition of human melanoma grafts in nude mice was dose-dependent, the inventors further examined the levels of the drug in mouse serum and tumor tissues following oral administration over time. Eight hours after mice received oral doses of 10, 25 and 75 mg/kg of BMS-345541, samples were collected and BMS-345541 was measured as described (Burke et al., 2003). BMS-345541 concentrations in serum reached 0.90±0.25, 3.1±1.1 and 8.2±1.1 μM in mice receiving 10, 25, 75 mg/kg BMS-345541, respectively. Tumor tissue levels of drug were 3.2±0.97, 12.5±3.28 and 28.1±2.65 nmoles per gram tissue protein for the three treatment groups, respectively. To determine levels of BMS-345541 over time in tumor-bearing mice, blood and tumor tissues were collected at times of 1.5, 3, 5, 8 and 24 h after an oral dose of 75 mg/kg. Results in FIG. 1E show that concentrations of BMS-345541 in serum were 6.6±0.31, 18±1.3, 5.2±0.19, 4.1±0.22, 1.1±0.065 μM and in tumor tissues were 19±2.5, 44±1.5, 30±1.0, 29±2.5, 11±0.83 nmoles/g tissue protein corresponding to time points of 1.5, 3, 5, 8, 24 h after drug administration.

Inhibition of IKK Activity Correlates with Suppression of Both NF-κB Activation and CXCL1 Production.

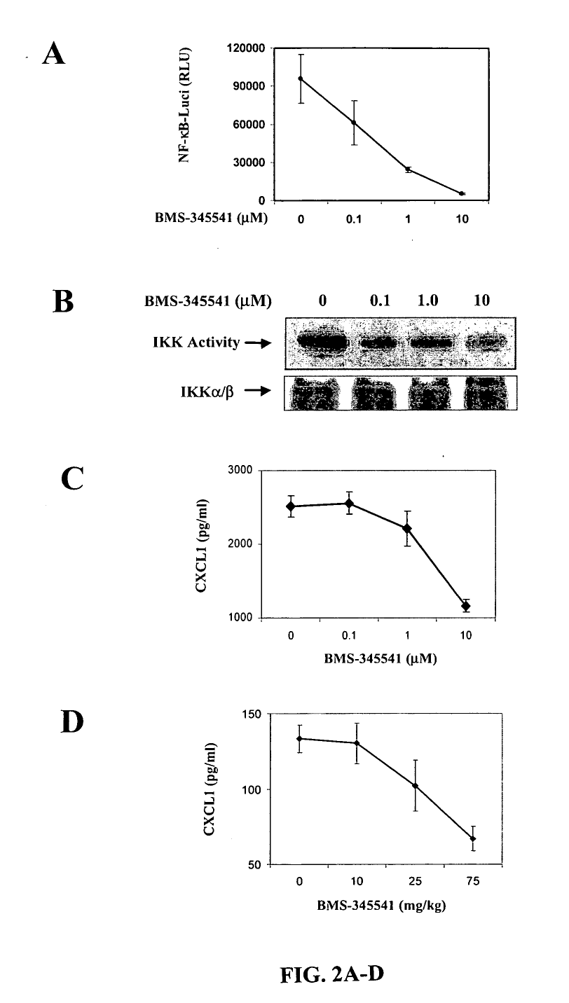

To determine whether BMS-345541 inhibition of melanoma cell proliferation is associated with the IKK/NF-κB signaling pathway, SK-Mel-5 melanoma cells were transfected with an NF-κB-dependent luciferase reporter gene and treated with BMS-345541 for an additional 36 h. The experiments revealed that the constitutive NF-κB activity in melanoma cells was dramatically reduced by BMS-345541 in a concentration dependent manner (FIG. 2A). BMS-345541 treatment (0.1, 1.0 and 10 μM) resulted in 36%, 75% and 95% reduction in NF-κB-dependent luciferase activity, respectively. To determine whether inhibition of NF-κB activation was the result of down-regulation of the upstream IKK activity by BMS-345541, SK-Mel-5 melanoma cells were treated with BMS-345541. Equal aliquots of cytoplasmic IKK proteins were immunoprecipitated with anti-IKKα/β antibodies and IKK activity was determined in an in vitro kinase assay using recombinant IκBα protein as a substrate for IκB kinase. The IKK activity was determined by autoradiography. The high activity of IKK in SK-Mel-5 melanoma cells was reduced by BMS-345541 in a concentration-dependent fashion (FIG. 2B). The inventors further explored whether washing could reverse BMS-345541 inhibition of IKK activity. In cell-free conditions, immunopurified IKK proteins were incubated with 10 μM BMS-345541 for 20 min and the IKK-containing beads were washed with kinase buffer three times prior to determination of the kinase activity. FIG. 2C shows that the BMS-345541-mediated inhibitory effect is reversed after the washing steps, indicating that this compound interacts with IKK proteins in a reversible manner.

The constitutive expression of CXCL1 by melanoma cells was speculated to produce an “autocrine loop” to promote melanoma tumorigenesis, since CXCL1 induces IKK activity in normal human melanocytes (Yang & Richmond, 2001). To determine whether BMS-345541 mediated-inhibition of IKK resulted in reduction of CXCL1 secretion, CXCL1 levels were determined by ELISA assay from the culture medium of melanoma cells and from serum of mice-bearing melanoma tumors treated or not treated with BMS-345541. The results in FIGS. 2C and 2D show that CXCL1 expression in melanoma cell cultures or mouse sera was suppressed in a concentration-dependent manner by BMS-345541 treatment. This is also in agreement with the decrease of NF-κB activity resulting from BMS-345541 treatment shown in FIG. 2A.

Blocking NF-κB/p65 Nuclear Translocation Leads to Melanoma Cell Apoptosis.

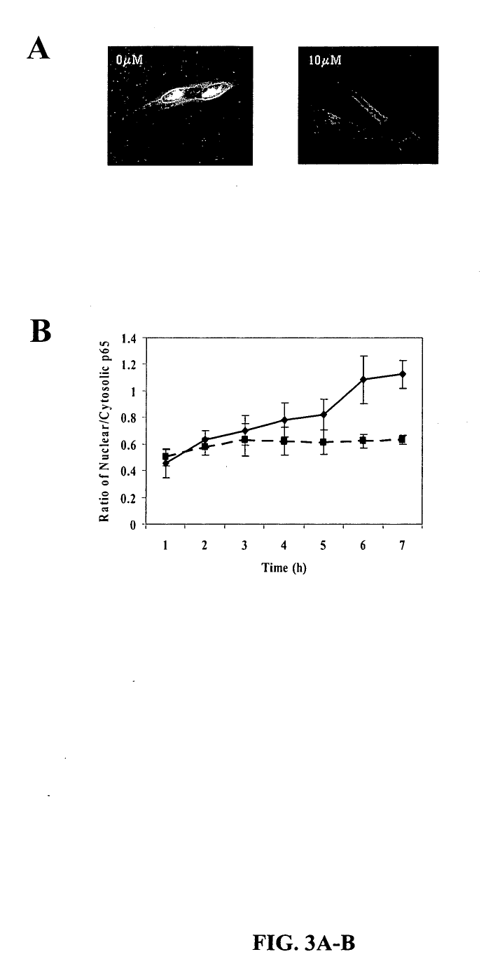

Since nuclear localization and activation of the NF-κB complex is often crucial for tumorigenesis (Sylla & Temin, 1986; Gilmore et al., 1996; White et al., 1996), particularly for human melanoma (Dhawan et al., 2002; Yang et al., 2001), inhibition of IKK activity in melanoma cells by BMS-345541 was speculated to stabilize the p65/p50 IκB complex in the cytoplasm and prevent NF-κB nuclear translocation and activation of gene expression. To test this possibility, SK-Mel-5 cells were transfected with a GFP-p65 expression vector to monitor the nuclear translocation in living cells. The GFP-fusion protein of p65 shows the same abilities as wild-type p65 for nuclear translocation, NF-κB-dependent promoter binding and functional transactivation of reporter genes (Schmid et al., 2000; Birbach et al., 2002). After overnight incubation following transfection with GFP-p65 DNA expression vector, cells were treated with DMSO vehicle as a control, or 10 μM of BMS-345541, one-hour prior to visualization by fluorescence microscopy. Time-lapse video microscopy was used to follow the localization of GFP-p65 in live cells. Images were captured at one-hour intervals over a 7 h observation period. The cytosol versus nuclear green fluorescent density was quantified by the Openlab software program. During the observation time of 7 h, in contrast to solvent control treatment, 10 μM of BMS-345541 treated melanoma cells consistently retained GFP-p65 in the cytoplasm (FIGS. 3A & B). After an exposure to 10 μM BMS-34541, SK-Mel-5 cells initiated apoptosis in response to inhibitor treatment (data not shown). Moreover, when the NF-κB super repressor (CFP-IκBαAA) and the expression vector YFP-p65 were cotransfected in SK-Mel-5 cells at a 9:1 ratio, induction of apoptosis was observed similar to that of cells treated with 10 μM BMS-34541 (data not shown).

Inhibition of Human Melanoma Tumor Growth by BMS-345541 is Through Apoptotic Pathways.

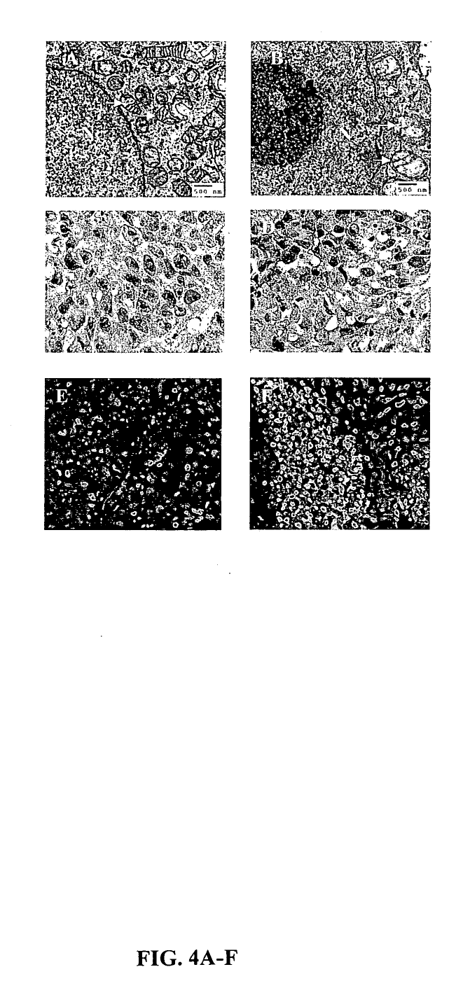

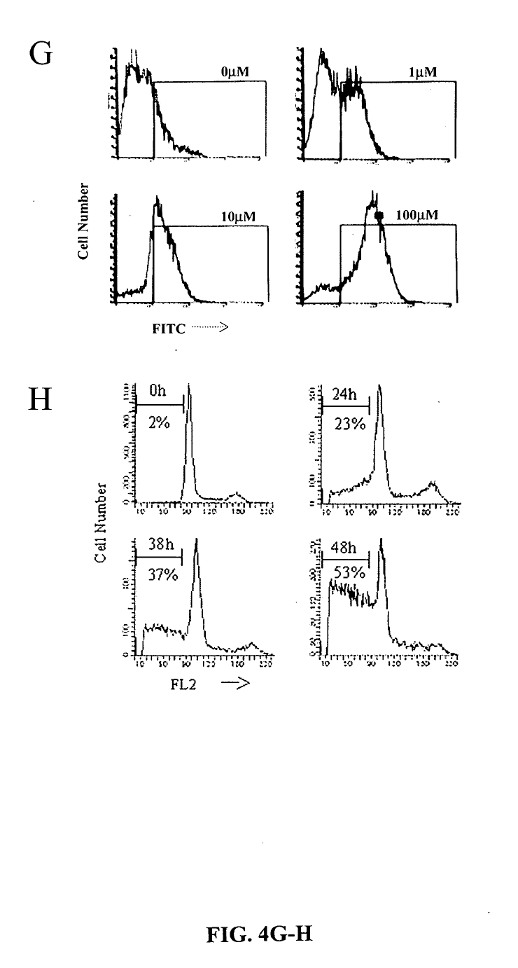

In melanoma, constitutive activation of NF-κB confers tumor survival capacity and escape from apoptosis (Richmond, 2002). The inventors hypothesized that BMS-345541 induced apoptosis of melanoma tumor cells through suppression of NF-κB activation. To characterize the mechanism for BMS-345541 suppression of melanoma tumor growth, cultured melanoma cells were exposed to 10 μM BMS-345541 for 24 h. These cells showed a typical feature of apoptosis (FIG. 4B) along with the altered mitochondria and nuclear chromatin condensation, in contrast to normal cells (FIG. 4A) under electron microscopy. The similar morphological phenomenon was observed in vivo. Melanoma tumor sections from mice treated with BMS-345541 were stained with H&E (FIGS. 4C & D) and for nuclear apoptosis using the DeadEnd Fluorometric TUNEL reagent. In contrast to the melanoma tissues treated with vehicle alone (FIGS. 4C & E), tumors treated daily with 25 mg/kg BMS-345541 (FIGS. 4D & F) showed obvious nuclear condensation and DNA fragment staining with fluorescent TUNEL. Moreover, when mice were treated with 75 mg/kg BMS-345541, TUNEL staining appeared extensively over the tumor sections (data not shown). To further quantitatively examine the drug-induced apoptotic melanoma cells, SK-Mel-5 melanoma cells were cultured in medium with BMS-345541 treatment for 36 h. Apoptotic cells were labeled using DeadEnd Fluorometric TUNEL reagent and analyzed by flow cytometry. Application of 1.0, 10 and 100 μM of BMS-345541 to SK-Mel-5 cell cultures induced a 2, 3.3 and 4 fold increase, respectively, in apoptotic cells when compared to the cells treated with a vehicle. Clearly, induction of apoptosis in melanoma cells by BMS-345541 appears to be concentration-dependent (FIG. 4G). To confirm that modulation of the IKK pathway leads to apoptosis in melanoma cells, DNA from dominant negative expression vectors for either IKKβ (K44M) or IκBα (S32, 36A) were independently transfected into SK-Mel-5 cells. Thirty-six hours after transfection, cells were collected, labeled with TUNEL reagent and analyzed by flow cytometry. In contrast to mock-transfected cells (vector-transfected control cells), the percentage of apoptotic melanoma cells in IKKβ (K44M) and IκBα (S32, 36A) transfected cells was 55.5% and 96.3% greater, respectively (data not shown). To examine nuclear apoptosis, SK-Mel-5 cells were exposed to 10 μM BMS-345541 for different time points. Induction of DNA fragmentation, a hallmark of apoptosis, was evaluated by staining DNA with ethidium bromide followed by quantization by FACS analysis. Induction of DNA fragmentation by BMS-345541 was both concentration-(data not shown) and time-dependent (FIG. 4H).

BMS-345541 Induces Mitochondria-Mediated Apoptosis.

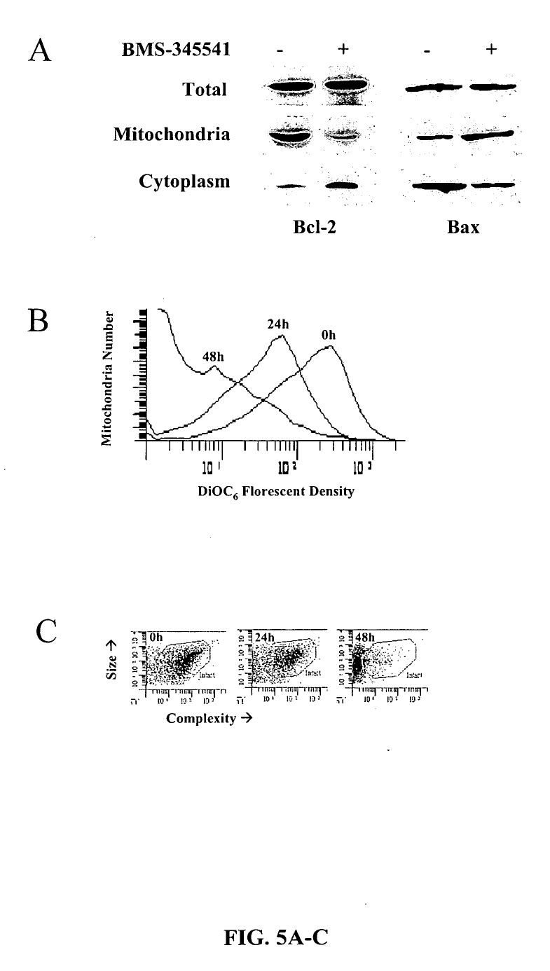

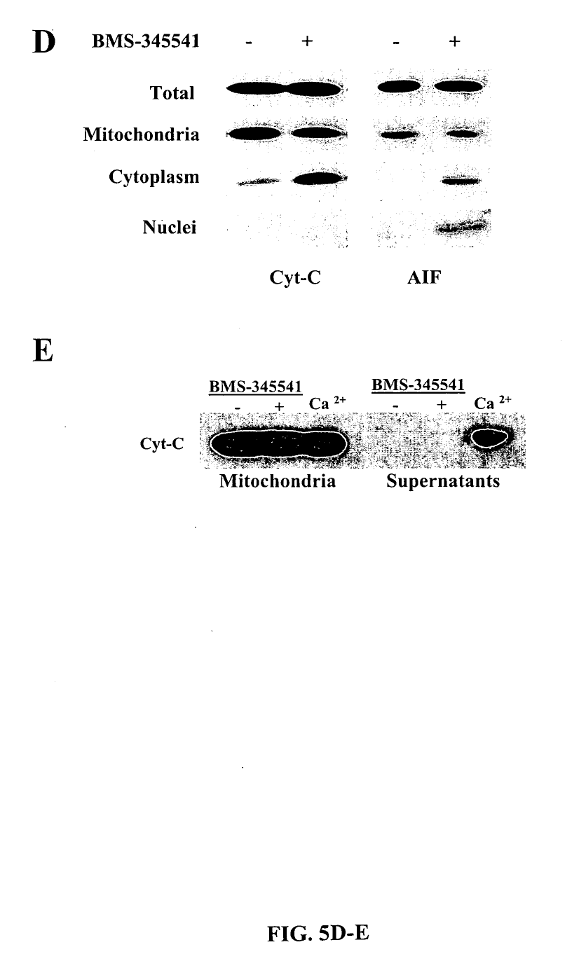

Mitochondria play a crucial role in the regulation of programmed cell death (Zamzami et al., 1995a; 1995b; 1996a; 1996b). The release of proteins from the intermembrane space of mitochondria is one of the pivotal events in the initiation of the apoptotic process (Henry-Mowatt et al., 2004). To investigate alteration of the apoptotic proteins in mitochondria during BMS-345541-induced apoptosis, the ratio of Bcl-2 and Bax distributed in mitochondria was analyzed in SK-Mel-5 cells using Western blot. A decreased ratio of Bcl-2 to Bax with BMS-345541 treatment at 24 h was observed (FIG. 5A). Either decreased Bcl-2 or increased Bax in mitochondria results in the instability of the mitochondrial membrane. The change in mitochondrial membrane potential (Δψm) was determined by FACS analysis after the mitochondria were stained with DiOC₆. SK-Mel-5 cells showed obvious loss of mitochondrial membrane potential when exposed to BMS-345541 for 24 h and 48 h (16±1.5% and 87±5.4%, respectively) (FIG. 5B). Reduction in the intact mitochondria population occurred after 48 h of 10 μM BMS-345541 treatment (FIG. 5C). Inhibition of NF-κB by over-expression of either IKKβ(KM) or the IκBα (S32, 36A) super-repressor in SK-Mel-5 melanoma cells resulted in a respective 32±16% or 52±2.8% loss of mitochondrial potential, in contrast to vector controls, confirming that NF-κB inhibition is the cause for mitochondrial dissipation. Moreover, 10 μM BMS-345541 treatment resulted in the important pro-apoptotic molecules such as mitochondrial cytochrome-c release into the cytoplasm and AIF nuclear translocation (FIG. 5D). To rule out a direct action of BMS-34554 on mitochondria, mitochondria isolated from SK-Mel-5 were treated with 10 μM BMS-345541, and the release of cytochrome-c into the supernatant was analyzed by Western blot (Boya et al., 2004). In contrast to DMSO negative control and the 200 μM Ca²⁺ positive control, no direct action of 10 μM BMS-345541 on mitochondria was observed, based on a failure to see release of cytochrome-c into the supernatant (FIG. 5E).

BMS-345541-Induced Apoptosis is Largely Caspases-Independent and AIF Dependent.

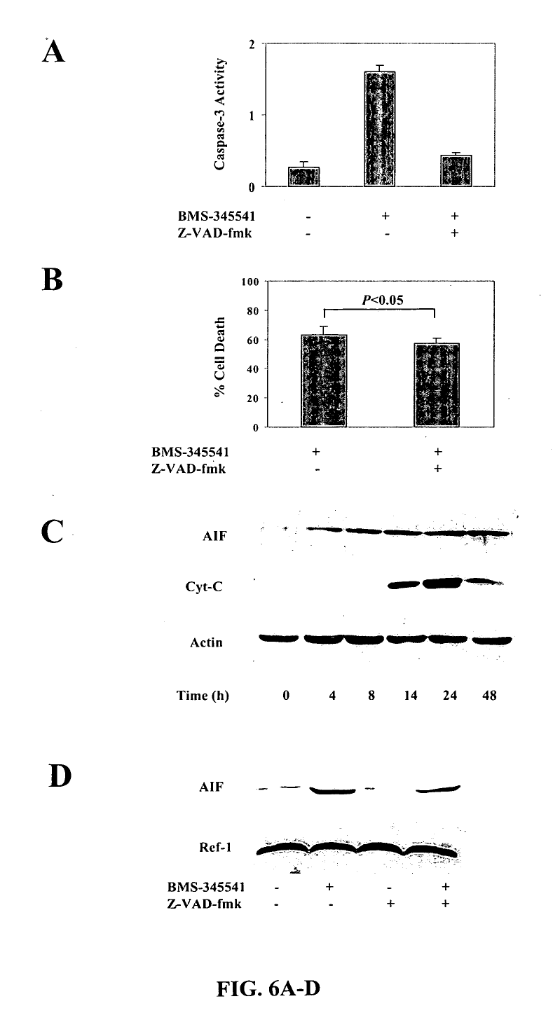

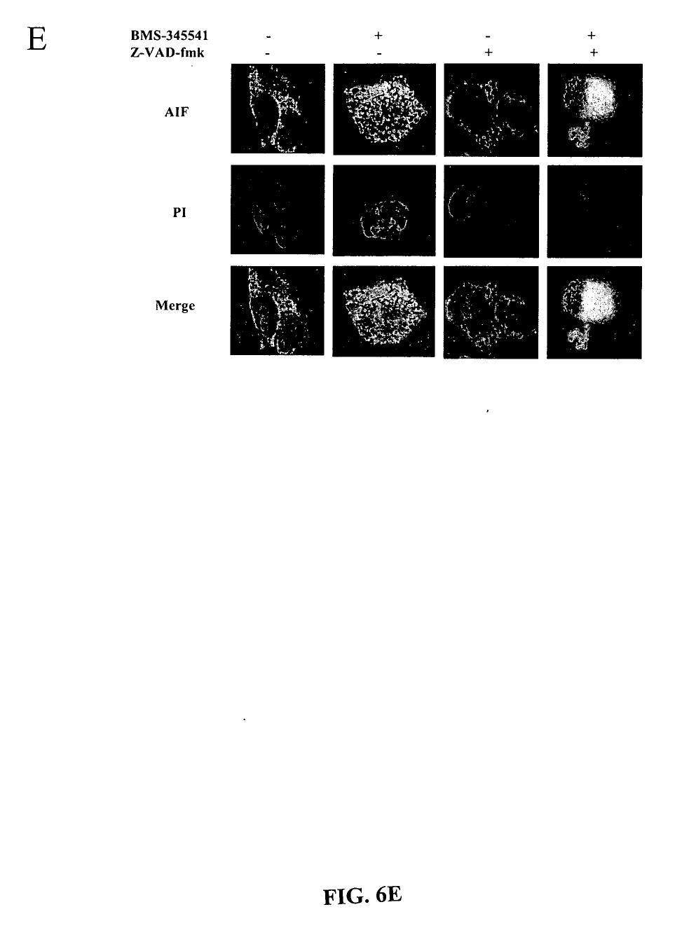

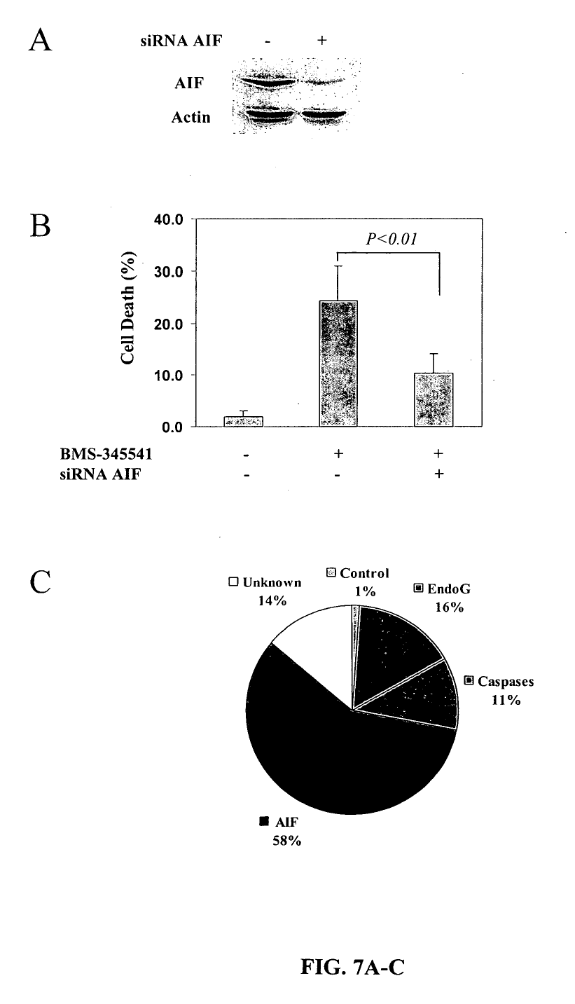

To examine whether BMS-345541 induction of apoptosis in melanoma cells is through a caspase dependent pathway, SK-Mel-5 melanoma cells were treated with 10 μM BMS-345541 for 36 h. Cytosol extracts were tested for caspase activity with the caspase-3 substrate, Ac-DEVD-pNA. The specific caspase-3 activity in response to treatment with vehicle DMSO, 10 μM BMS-345541 or combination with 50 μM Z-VAD-fmk was measured to levels of 0.27±0.075, 1.6±0.092 and 0.44±0.035 pmol/min/μg protein respectively (FIG. 6A). These results indicate that BMS-345541 is able to induce caspase-3 activity in cytosolic extracts and this induction can be blocked by 87.2% with the pan-caspase inhibitor Z-VAD-fink. The cell permeable pan-caspase inhibitor, Z-VAD-fink, is able to block caspase-dependent apoptosis in a number of melanoma cell lines (Zhang et al., 2004). However, when SK-Mel-5 cells were treated with 50 μM Z-VAD-fink for 48 h, the pan-caspase inhibitor protected only 11% (p<0.05) of cells from nuclear apoptosis induced by 10 μM BMS-345541 (FIG. 6B). These data indicate that BMS-345541 induced apoptosis is not prevented by Z-VAD-fmk and therefore functions through a pathway that is largely caspase-independent. AIF is a mitochondria-localized flavoprotein and is known to be involved in apoptosis via a caspase-independent pathway. To determine whether AIF was involved BMS-345541-induced apoptosis, the release of AIF and cytochrome-c into the cytosol was evaluated at different time points after exposure of SK-Mel-5 cells to 10 μM BMS-345541. Data in FIG. 6C show an initiation of AIF release at 4 h, whereas cytochrome-c was not released until 14 h after BMS-345541 treatment. These data implicate an involvement of AIF in the initiation of the apoptotic signal and this is followed by cytochrome-c release. To rule out the possibility that AIF is also involved in a caspase-dependent apoptotic pathway (Arnoult et al., 2002; 2003), SK-Mel-5 cells were treated with 10 μM BMS-345541 and/or 50 μM Z-VAD-fmk, and AIF nuclear translocation was examined by Western blot and confocal microscopy. As shown in FIGS. 6D & E, the pan-caspase inhibitor Z-VAD-fink failed to influence AIF translocation to nucleus and nuclear condensation. Thus, BMS-345541 induced AIF nuclear translocation and nuclear apoptosis in a caspase-independent manner. Moreover, RNAi targeted against AIF protein by over 90% knockdown after three successive transfections of 100 nM of siRNA (FIG. 7A), effectively reduced BMS-345541-induced the rate of apoptotic cells from 24.3±6.8% to 10.3±3.8% (FIG. 7B). In addition, siRNA targeting of EndoG using the same approach knocked down over 90% of total EndoG protein, which resulted in a 16% (p<0.05) attenuation of BMS-345541-induced cell death (data not shown) in contrast to 58% (p<0.01) apoptotic attenuation by siRNA AIF (FIG. 7C). Importantly, translocation of mitochondrial AIF to nucleus is a critical step for BMS-345541-induced caspase-independent cell death in Sk-Mel-5 melanoma cells. However, approximate 14% of apoptosis induced by BMS-345542 has not been accounted for in our experiments.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,452,901
U.S. Pat. No. 6,235,740
U.S. Pat. No. 6,239,133
U.S. Pat. No. 6,635,626
U.S. Patent Pub. 20030022898
Arap et al., Cancer Res., 55(6):1351-1354, 1995.
Arnoult et al., Cell Death Differ., 10(7):845-849. 2003.
Arnoult et al., J. Cell Biol., 159(6):923-929, 2002.
Bakhshi et al., Cell, 41(3):899-906, 1985.
Baldwin, Jr., Annu. Rev. Immunol., 14:649-683, 1996.
Beg et al., Genes Dev., 6:1899-1913, 1992.
Birbach et al., J. Biol. Chem., 277:10842-10851, 2002.
Boya et al., Biochim. Biophys. Acta., 1659(2-3):178-189, 2004.
Burke et al., J. Biol. Chem., 278:1450-1456, 2003.
Cahir-McFarland et al., Proc. Natl. Acad. Sci. USA, 97:6055-6060, 2000.
Caldas et al., Nat. Genet., 8(1):27-32, 1994.
Chawla-Sarkar et al., J. Biol. Chem., 278:39461-39469, 2003.
Cheng et al., Cancer Res., 54(21):5547-5551, 1994.
Cleary and Sklar, Proc. Natl. Acad. Sci. USA, (21):7439-7443, 1985.
Cleary et al., J. Exp. Med., 164(1):315-320, 1986.
Dhawan et al., Cancer Res., 62:7335-7342, 2002.
Frankfurt et al., Exp. Cell Res., 226(2):387-397, 1996.
Frick et al., Hautarzt, 53:659-665, 2002.
Ghosh et al., Annu. Rev. Immunol., 16:225-260, 1998.
Gilmore et al., Oncogene, 13:1367-1378, 1996.
Haghnegahdar et al., J. Leukoc. Biol., 67:53-62, 2000.
Henry-Mowatt et al., Oncogene, 23(16):2850-2860, 2004.
Hollstein et al., Science, 253(5015):49-53, 1991.
Hussussian et al., Nat. Genet., 8(1):15-21, 1994.
Huxford et al., Cell, 95:759-770, 1998.
Kamb et al., Nat. Genet., 8(1):23-26, 1994.
Keller et al., Blood, 96:2537-2542, 2000.
Kerr et al., Br. J. Cancer, 26(4):239-257, 1972.
Liptay et al., Int. J. Cancer, 105:735-746, 2003.
Malek et al. J. Biol. Chem., 273:25427-25435, 1998.
Maniatis, Science, 278:818-819, 1997.
Margolin et al., J. Cancer Res. Clin. Oncol., 128:214-218, 2002.
Mathas et al., Blood, 102:1028-1034, 2003.
Mercurio et al., Science, 278:860-866, 1997.
Moore and Bose, Virology, 162:377-387, 1988.
Mori et al., Cancer Res., 54(13):3396-3397. 1994.
Nobri et al., Nature (London), 368:753-756, 1995.
Okamoto et al., Proc. Natl. Acad. Sci. USA, 91(23):11045-11049, 1994.
Orlow et al., Int. J. Oncol., 15(1):17-24, 1994.
Regnier et al., Cell, 91:243-252, 1997.
Richmond et al., Cancer Res., 45:6390-6394, 1985.
Richmond, Nat. Rev. Immunol., 2:664-674, 2002.
Rodriguez et al., Mol. Cell Biol., 15:2413-2419, 1995.
Sambrook et al., In: Molecular cloning, Cold Spring Harbor Laboratory Press, Cold Spring Harbor, N.Y., 2001.
Schmid et al., J. Biol. Chem., 275:17035-17042, 2000.
Serrano et al., Nature, 366:704-707, 1993.
Serrano et al., Science, 267(5195):249-252, 1995.
Shattuck-Brandt and Richmond, Cancer Res., 57:3032-3039, 1997.
Smalley and Eisen, Int. J. Cancer, 105:165-175, 2003.
Spencer et al., Genes Dev., 13:284-294, 1999.
Spiro et al., Forum (Genova), 10:274-285, 2000.
Strauss et al., Br. J. Cancer, 89:1901-1905, 2003.
Streit and Detmar, Oncogene, 22:3172-3179, 2003.
Sylla and Temin, Mol. Cell Biol., 6:4709-4716, 1986.
Tsujimoto and Croce, Proc. Natl. Acad. Sci. USA, 83(14):5214-5218, 1986.
Tsujimoto et al., Science, 228(4706):1440-1443, 1985.
Wang et al., Science, 274:784-787, 1996.
Weinberg, Science, 254(5035):1138-1146, 1991.
White et al., Mol. Cell Biol., 16:1169-1178, 1996.
Woronicz et al., Science, 278:866-869, 1997.
Yang and Richmond, Cancer Res., 61:4901-4909, 2001.
Yang et al., Cancer Res., 61:8150-8157, 2001.
Yaron et al., Nature, 396:590-594, 1998.
Zamzami et al., FEBS Lett., 384(1):53-57, 1996a.
Zamzami et al., J. Exp. Med., 181(5):1661-1672, 1995a.
Zamzami et al., J. Exp. Med., 182(2):367-377, 1995b.
Zamzami et al., J. Exp. Med., 183(4):1533-1544, 1996b.
Zandi and Karin, Mol. Cell Biol., 19:4547-4551, 1999.
Zandi et al., Cell, 91:243-252, 1997.
Zhang et al, Mol. Cancer Ther., 3(4):425-435, 2004.

What is claimed is:

1. A method of inhibiting growth of a melanoma cancer cell comprising contacting the cell with (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or an analog, or a salt thereof.

2. The method of claim 1, wherein inhibiting comprises inducing apoptosis in the melanoma cancer cell.

3. The method of claim 1, wherein inhibiting comprises inducing cell cycle arrest in the melanoma cancer cell.

4. The method of claim 1, wherein inhibiting comprises inducing cell stasis in the melanoma cancer cell.

5. The method of claim 1, wherein the melanoma cancer cell is located in a cell culture.

6. The method of claim 1, wherein the melanoma cancer cell is located in a tissue culture.

7. The method of claim 1, wherein the melanoma cancer cell is located in a mammal.

8. The method of claim 7, wherein the mammal is a human.

9. The method of claim 1, wherein the melanoma cancer cell is a premalignant melanoma cancer cell.

10. The method of claim 1, wherein the melanoma cancer cell is a malignant melanoma cancer cell.

11. The method of claim 1, wherein the melanoma cancer cell is a metastatic melanoma cancer cell.

12. The method of claim 1, wherein the melanoma cancer cell is a multidrug resistant melanoma cancer cell.

13. A method of inhibiting NFκB activity in a melanoma cancer cell comprising providing to the cell (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or an analog, or a salt thereof.

14. The method of claim 13, further comprising inhibiting expression of NFκB.

15. The method of claim 13, wherein inhibiting comprises inducing apoptosis in the melanoma cancer cell.

16. A method of inhibiting tumorigenesis of a melanoma cancer cell comprising contacting the cell with an effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or an analog or a salt thereof.

17. A method for treating or preventing melanoma in a subject comprising administering to the subject a therapeutically effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or an analog or a salt thereof to inhibit cell proliferation.

18. The method of claim 17, comprising inducing cytotoxicity in the melanoma cancer cell.

19. The method of claim 17, comprising inducing apoptosis in the melanoma cancer cell.

20. The method of claim 17, wherein the melanoma is a premalignant melanoma cancer.

21. The method of claim 17, wherein the melanoma is a malignant melanoma cancer.

22. The method of claim 17, wherein the melanoma is a metastatic melanoma cancer.

23. The method of claim 17, wherein the melanoma cancer cell is a multidrug resistant melanoma cancer cell.

24. The method of claim 17, further comprising providing a second therapeutic agent.

25. The method of claim 24, wherein the second therapeutic agent is a chemotherapeutic agent, a radiotherapeutic agent, an immunotherapeutic agent or a gene therapy.

26. The method of claim 24, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof is provided to the subject before the second therapeutic agent.

27. The method of claim 24, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof is provided to the subject after the second therapeutic agent.

28. The method of claim 24, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof is provided to the subject at the same time as the second therapeutic agent.

29. The method of claim 17, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof is provided more than once.

30. The method of claim 24, wherein the second therapeutic agent is provided more than once.

31. The method of claim 24, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof in combination with a second therapeutic agent is provided more than once.

32. The method of claim 24, wherein (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof and the second therapeutic agent are provided to a subject intratumoraly, intravenously, intraperitoneally, intramuscularly, orally, or by inhalation.

33. A method for assaying for the inhibition of melanoma cancer cell growth comprising:
a) providing a melanoma cancer cell sample;
b) contacting the cell sample with an effective amount of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof;
c) analyzing the cell sample for growth inhibition; and,
d) comparing the inhibition of the cell growth in step (c) with the inhibition of a melanoma cancer cell in the absence of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt thereof, wherein the difference in growth inhibition represents the growth inhibitory effect of (4(2′-aminoethyl)amino-1,8-dimethylimididazo(1,2-a)quinoxaline)-4,5-dihydro-1,8-dimethylimidazo(1,2-a)quinoxalin-4-one-2-carboxylic acid or pharmaceutically acceptable salt, or analog thereof.

34. The method of claim 33, wherein growth inhibition is analyzed by MTT assay.

35. The method of claim 33, wherein growth inhibition is analyzed by cell number count assay.

36. The method of claim 33, further comprising analyzing the sample for induction of apoptosis.

37. The method of claim 36, wherein induction of apoptosis is analyzed by FACS.

38. The method of claim 36, wherein induction of apoptosis is analyzed by TUNEL assay.

39. The method of claim 33, further comprising analyzing the sample for inhibition or reduction of IKK activity.

40. The method of claim 33, further comprising analyzing the sample for NFκB/p65 nuclear translocation.

41. The method of claim 33, further comprising analyzing the melanoma cancer cell sample for reduction of expression of a chemokine.

42. The method of claim 41, wherein the chemokine is CXCL1 or CXCL8.


SEQUENCE LISTING (4 sequences)

SEQ ID NO: 1 - DNA, 30 bp, Artificial Sequence
  Description of Artificial Sequence Synthetic Primer
ttcgaattctatgttccagcccgcagagcc

SEQ ID NO: 2 - DNA, 30 bp, Artificial Sequence
  Description of Artificial Sequence Synthetic Primer
tgcggtacctcataacgtcaggcgctggcc

SEQ ID NO: 3 - DNA, 33 bp, Artificial Sequence
  Description of Artificial Sequence Synthetic Primer
ttcgaattctagctggtcaccttccctgacaac

SEQ ID NO: 4 - DNA, 29 bp, Artificial Sequence
  Description of Artificial Sequence Synthetic Primer
tcgggtaccatgcatgctcgagcggccgc